(19) United States
(12) Patent Application Publication
(10) Pub. No.: US 2007/0179590 A1
(43) Pub. Date: Aug. 2, 2007

(54) Hybrid intraluminal device with varying expansion force

(76) Inventors: Wenfeng Lu, Pfafftown, NC (US); Brian K. Rucker, King, NC (US)

Correspondence Address: BRINKS HOFER GILSON & LIONE, P.O. BOX 10395, CHICAGO, IL, 60610, US

(21) Appl. No.: 11/646,798

(22) Filed: Dec. 28, 2006

Related U.S. Application Data
(60) Provisional application No. 60/754,742, filed on Dec. 29, 2005.

(51) Int. Cl. A61F 2/90; A61F 2/06

(52) U.S. Cl. 623/1.16; 623/1.31; 623/1.53

(57) ABSTRACT

A hybrid intraluminal device and a method for fabricating the device is described. The device has structural elements which have different properties. One portion of the device may contain a stent having a zigzag configuration. A second portion of the device may contain a stent having a braided configuration. The first and second portions may possess the same architectural pattern but yet exhibit variation in radial force as a result of various properties of the structural elements. The portions are attached to a coating to form a hybrid stent. Gaps between the different stent sections provide flexibility to the stent. The first and second portions may be configured in numerous ways. The structural features of the hybrid stent can be adapted to satisfy the criteria of specific medical applications.

### RELATED APPLICATIONS

This application claims the benefit of priority from U.S. Provisional Application No. 60/754,742 filed Dec. 29, 2005, which is incorporated herein by reference.

### FIELD OF THE INVENTION

The invention generally relates to a stent having a combination of different structural elements.

### BACKGROUND

Stents are utilized in a variety of medical procedures. They can be placed within numerous regions of the body, including the esophagus, bile duct, pancreatic duct, small intestine, and vasculature. The design features of a stent must be modified in accordance with the type of medical procedure to be performed and the area of the body the stent is to be implanted within.

Numerous stent designs are currently available. For example, one group of stents, known as zigzag shaped stents, have a zig-zag configuration which can provide relatively large expansive radial forces against a body lumen. Such large radial forces can fixate the stent at a target region, thereby reducing the likelihood of stent migration. Moreover, such stents can sufficiently collapse into a compressed state during delivery. Upon deployment, the zigzag shaped stents are capable of expanding without undergoing a reduction in length (i.e., foreshortening). However, the rigid shape of such zigzag shaped stents translates into poor flexibility. Accordingly, zigzag shaped stents do not perform well when implanted in curved body lumens.

To overcome the inherent lack of flexibility of the zigzag shaped stents, braided stents have also been utilized. The braided geometry of a braided stent provides the needed flexibility to accommodate curved body lumens. The woven design prevents the braided stent from kinking. However, braided stents expand with relatively small radial force against a body lumen. Such a relatively small radial force is frequently too weak to hold a body lumen open. The small radial force can also lead to stent migration. Additionally, expansion of a braided stent causes significant foreshortening of the stent as a result of its interwoven structure.

Moreover, current stent designs exhibit large radial forces at the end portions of the stent to prevent migration of the stent. The large radial forces provided by current stent designs have demonstrated the ability to fixate the stent at the desired implantation site. However, the large radial forces along the end portions of the stent have also shown a tendency to irritate tissue, thereby stimulating the tissue to grow rapidly around the ends of the stent. Such tissue overgrowth is commonly known as hyperplasia and may lead to in-stent restenosis.

In view of the drawbacks of current stent designs, there is an unmet need for an improved stent that can provide a radial force against a body lumen which is sufficiently large to prevent migration but not excessively large to stimulate adverse tissue overgrowth. Moreover, the improved stent would provide flexibility to permit implantation in curved body lumens, preferably without undergoing significant foreshortening upon expansion.

### SUMMARY

Accordingly, a hybrid stent is provided with a combination of different structural properties.

In a first aspect, an intraluminal device is provided having a cylindrical body. The cylindrical body has a first expandable stent structure and a second expandable stent structure. The first expandable stent structure has a radial expanding force that is different than the second expandable stent structure.

In a second aspect, an intraluminal device is provided having a generally cylindrical body. The cylindrical body includes a body portion, a first end portion and a second portion. The body portion includes zigzag shaped stents having an outer body diameter. Each of the zigzag shaped stents are longitudinally spaced apart without being interconnected to each other. The zigzag shaped stents are disposed circumferentially around the cylindrical body and extend along a portion of a longitudinal axis of the cylindrical body. The end portions include a flexible element. The end portions have an outermost diameter greater than the outer body diameter of the zigzag shaped stents. The end portions extend in a helical pattern along a portion of the longitudinal axis to form a braided configuration. A coating is attached to the body portion and the end portions.

In a third aspect, an intraluminal device is provided having a generally cylindrical body which includes a body portion, a first end portion and a second end portion. The body portion has a flexible element extending in a helical pattern along a portion of the longitudinal axis of the cylindrical body to form a braided configuration. The braided configuration has an outer diameter. The end portions include zigzag shaped structural members that are disposed circumferentially around the cylindrical body and extend along a portion of the longitudinal axis of the cylindrical body. The end portions have a diameter greater than the outer diameter of the body portion. A coating attaches to the body portion and the end portions.

### BRIEF DESCRIPTION OF THE DRAWINGS

Embodiments will now be described by way of example with reference to the accompanying drawings, in which:

FIG. 1 is a side view of a hybrid stent in an expanded state with a zigzag configuration at the proximal and distal portions and a braided stent configuration at the body portion;

FIG. 2 is a side view of a hybrid stent in an expanded state with a braided configuration at the proximal and distal portions and zigzag cages along the body portion;

FIG. 3 is a side view of a hybrid zigzag stent in an expanded state with a covering extending a predetermined distance beyond the portions of the stent;

FIG. 4 is a side view of a hybrid stent in an expanded state with asymmetrical proximal and distal portions; and

FIG. 5 is a flow schematic of a process for manufacturing a hybrid stent.

FIG. 6 is a side view of a hybrid braided stent with flanged ends;

FIG. 7 is a side view of a hybrid braided stent with dumbbell shaped ends;

FIG. 8 is a plot of radial force along the length of the stent of FIG. 6;

FIGS. 9, 10 are plots of radial force along the length of conventional stents;

FIG. 11 is a side view of a hybrid stent with braided elements that intersect to form various sized junctions along the length of the stent;

FIG. 12 is a junction of the stent of FIG. 11 along the end portions;

FIG. 13 is a junction of the stent of FIG. 11 along the center portion;

FIG. 14 is a side view of a body cage portion of a braided hybrid stent in which a first group of filaments are collectively wound in a first helical direction and a second group of filaments are collectively wound in a second helical direction.

### DETAILED DESCRIPTION OF THE PREFERRED EMBODIMENTS

The embodiments are described with reference to the drawings in which like elements are referred to by like numerals. The relationship and functioning of the various elements of the embodiments are better understood by the following detailed description. However, the embodiments as described below are by way of example only, and the invention is not limited to the embodiments illustrated in the drawings. It should also be understood that the drawings are not to scale and in certain instances details have been omitted, which are not necessary for an understanding of the embodiments, such as conventional details of fabrication and assembly.

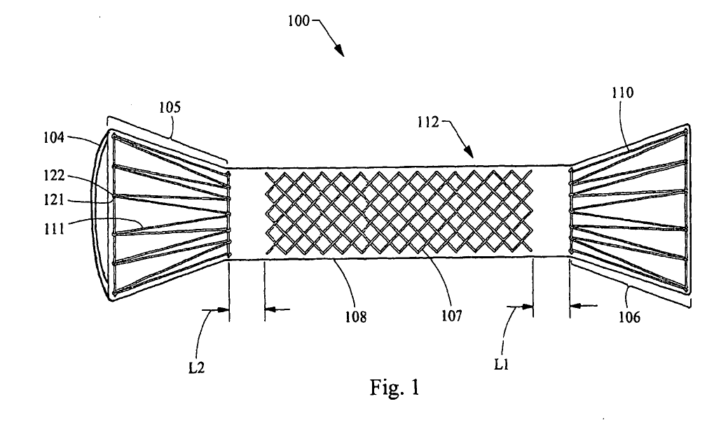

An exemplary hybrid stent is shown in FIG. 1. FIG. 1 shows a hybrid stent 100 with a combination of zigzag stent and braided stent elements. The hybrid stent 100 has a proximal portion 105, a body portion 112, and a distal portion 106. Generally speaking, the combination of a braided design at the body portion 112 with zigzag stent cages at the proximal and distal portions 105, 106 results in a hybrid stent 100 that can be implanted within a curved body lumen and that can provide relatively high radial force against the curved body lumen. The term “zigzag” as used herein refers to any generally undulating pattern and includes segments which are connected by bends that are angled or rounded. The segments may straight or curvilinear. The term “braided” as used herein refers to any general woven pattern that includes segments which overlap in an interwoven arrangement.

The proximal and distal portions 105, 106 have respective structural members 111 and 110 extending circumferentially in a zigzag orientation to form zigzag cages. Although not shown, another set of zigzag structural members may be overlayered above members 111 and 110, optionally being offset from members 111 and 110. Each of the zigzag cages at the proximal and distal portions 105, 106 may be formed from a monofilament wire which is shaped into a zigzag configuration. The zigzag cages may be similar to the zigzag described in U.S. Pat. No. 4,580,568, which is incorporated herein by reference. The zigzag cages may be formed from any suitable metallic alloy such as stainless steel, nitinol or any other suitable biocompatible material. The shape of the zigzag cages include a series of straight sections 111 joined by bent portions or cusps 122. Each bend or cusp 122 defines an eye 121, which may be shaped by bending the wire. As explained below, the eye 121 may be used to secure the zigzag cage to a coating 108. The zigzag stents may be formed by any other method known to one of ordinary skill in the art, including laser cutting from a cannula.

The zigzag cages of the proximal and distal portions 105, 106 provide a relatively large radial force against a body lumen as compared with other stent designs. Such a large body radial force anchors the hybrid stent 100 in the desired region of a body lumen and prevents the hybrid stent 100 from migrating. To assist in anchoring the hybrid stent 100, proximal and distal portions 105, 106 may be flared, as shown in FIG. 1. Although FIG. 1 shows the proximal and distal portions 105, 106 with symmetrical flared portions, other variations are contemplated. For example, the flared portions may be cup-shaped, bell-shaped, or sphere-shaped. Additionally, the proximal and/or distal portions 105, 106 may have an abrupt step increase from the smaller diameter of the body portion 112 to a larger predetermined diameter. The proximal and distal portions 105, 106 may be symmetrical or asymmetrical. The particular geometry of the proximal and distal portions 105, 106 will be dependent upon a number of factors, including the site of implantation, the length of the stricture, and the relative tendencies of the proximal and distal ends to migrate.

The body portion 112 comprises a woven braided tubular structure. The braided tubular structure of the body portion 1 12 has flexible elastic elements 107, thereby making the hybrid stent 100 capable of being maneuvered through tortuous body lumens and being implanted in curved body lumens. The body portion 112 may be formed from single or multiple wires. Various methods of hand weaving or machine weaving, as are known by one of ordinary skill in the art may be used. For example, a mandrel having a diameter corresponding to the chosen diameter of the body portion 112 may be used as a support element. A single wire or multiple wires may then be helically woven along the surface of the mandrel to form a braided configuration. The wires may be bent around pins or tabs projecting from the mandrel. This allows the wires to cross each other to form a plurality of angles. A conventional braiding machine may also be utilized to arrange a single wire or multiple wires in a plain weave to form the braideded configuration of the body portion 112.

The ends of the single wire or multiple wires of the body portion 112 may be coupled together by using any suitable method known to one of ordinary skill in the art that is capable of preventing the wires from returning to their straight, unbent configuration. For example, the portions of the single or multiple wires may be bent and crimped within a metal clip. Additionally, the ends of the single or multiple wires may be coupled to each other by twisting, crimping or tying.

Suitable materials for the braided body portion 112 include any biocompatible material including shape memory metals. Preferably, nitinol is used.

The length and diameter of the body portion 1 12 will be dependent upon various factors, including the location within the patient's body where the stent 100 is to be implanted, and the length and geometry of the stricture. Suitable ranges of the length of the body portion 112 include from about 10 mm to about 130 mm, preferably from about 30 mm to about 110 mm, and most preferably from about 40 mm to about 100 mm. Suitable ranges of diameters for the body portion 112 include from about 14 mm to about 22 mm for an esophageal/enteral stent and from about 6 mm to about 12 mm for a biliary stent.

Still referring to FIG. 1, a coating 108 overlies the proximal portion 105, the distal portions 106, and the body portion 112. The coating 108 is continuous, extending the entire length of the hybrid stent 100. Although not shown, the coating 108 may be discontinuous such that sections of the body portion and/or proximal and distal portions 105, 106 are uncoated. The coating 108 attaches to the body portion 112 and the proximal, distal portions 105, 106. The coating may eliminate the need for direct attachment between the body portion 112 and the proximal, distal portions 105, 106 via interconnectors. Although FIG. 1 shows the coating as the sole means of attachment for the body portion 112 and the proximal, distal portions 105, 106, direct attachment to each other via interconnectors may be provided. Variations of a coating are contemplated, such as a cover or sleeve.

Any suitable biocompatible material may be used for the coating, including silicone, polyurethane, or combinations thereof. For example, a biocompatible polyurethane called THORALON may be utilized. THORALON is available from THORATEC in Pleasanton, Calif. THORALON has been used in certain vascular applications and is characterized by thromboresistance, high tensile strength, low water absorption, low critical surface tension and good flex life. THORALON and methods of manufacturing this material are disclosed in U.S. Pat. Application Publication No. 2002/0065552 A1 and U.S. Pat. Nos. 4,861,830 and 4,675,361, each of which is incorporated herein by reference in their entirety. As disclosed in these patents, THORALON is a polyurethane based polymer (referred to as BPS-215) blended with a siloxane containing surface modifying additive (referred to as SMA-300). Base polymers containing urea linkages can also be used. The concentration of the surface modifying additive may be in the range of 0.5% to 5% by weight of the base polymer.

THORALON can be manipulated to provide either a porous or non-porous material. Formation of porous THORALON is described, for example, in U.S. Pat. No. 6,752,826 and U.S. Pat. Application Publication No. 2003/0149471 A1, both of which are incorporated herein by reference in their entirety. The pores in the polymer may have an average pore diameter from about 1 micron to about 400 microns. Preferably the average pore diameter is from about 1 micron to about 100 microns, and more preferably is from about 1 micron to about 10 microns.

A variety of other biocompatible polyurethanes/polycarbamates and urea linkages (hereinafter “—C(O)N or CON type polymers”) may also be employed as the coating 108. Biocompatible CON type polymers modified with cationic, anionic and aliphatic side chains may also be used. See, for example, U.S. Pat. No. 5,017,664, which is incorporated herein by reference in its entirety. Other biocompatible CON type polymers include: segmented polyurethanes, such as BIOSPAN; polycarbonate urethanes, such as BIONATE; polyetherurethanes, such as ELASTHANE (all available from POLYMER TECHNOLOGY GROUP, Berkeley, Calif.); siloxane-polyurethanes, such as ELAST-EON 2 and ELAST-EON 3 (AORTECH BIOMATERIALS, Victoria, Australia); polytetramethyleneoxide (PTMO) and polydimethylsiloxane (PDMS) polyether-based aromatic siloxane-polyurethanes, such as PURSIL-10, -20, and -40 TSPU; PTMO and PDMS polyether-based aliphatic siloxane-polyurethanes, such as PURSIL AL-5 and AL-10 TSPU; aliphatic, hydroxy-terminated polycarbonate and PDMS polycarbonate-based siloxane-polyurethanes, such as CARBOSIL-10, -20, and -40 TSPU (all available from POLYMER TECHNOLOGY GROUP). Examples of siloxane-polyurethanes are disclosed in U.S. Pat. Application Publication No. 2002/0187288 A1, which is incorporated herein by reference in its entirety. In addition, any of these biocompatible CON type polymers may be end-capped with surface active end groups, such as, for example, polydimethylsiloxane, fluoropolymers, polyolefin, polyethylene oxide, or other suitable groups. See, for example the surface active end groups disclosed in U.S. Pat. No. 5,589,563, which is incorporated herein by reference in its entirety.

Other biocompatible polymeric materials may be used including poly(ethylene glycol) (PEG), polyanhydrides, polyorthoesters, fullerene, polytetrafluoroethylene, poly(styrene-b-isobutylene-b-styrene), polyethylene-co-vinylacetate, poly-N-butylmethacrylate, amino acid-based polymers (such as poly(ester) amide), SiC, TiNO, Parylene C, heparin, porphorylcholine.

Other polymeric materials include polyesters, poly(meth)acrylates, polyalkyl oxides, polyvinyl alcohols, polyethylene glycols, polyvinyl pyrrolidone, and hydrogels. Other polymers that may be dissolved and dried, cured or polymerized on the stent may also be used. Such polymers include, but are not limited to: polyolefins, polyisobutylene and ethylene-alphaolefin copolymers; acrylic polymers (including methacrylate) and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics; copolymers of vinyl monomers with each other and olefins; polyamides; alkyd resins; polycarbonates; polyoxymethylenes; polyimides; polyethers; epoxy resins; polyurethanes; rayon; rayon-triacetate; cellulose; cellulose acetate; cellulose butyrate; cellulose acetate butyrate; cellophane; cellulose nitrate; cellulose propionate; cellulose ethers; and modifications, copolymers, and/or mixtures of any of the carriers identified herein. The polymers may contain or be coated with substances that promote endothelialization and/or retard thrombosis and/or the growth of smooth muscle cells.

Additionally, the coating may be a hydrophilic polymer. The hydrophilic polymer may be selected from the group comprising polyacrylate, copolymers comprising acrylic acid, polymethacrylate, polyacrylamide, poly(vinyl alcohol), poly(ethylene oxide), poly(ethylene imine), carboxymethylcellulose, methylcellulose, poly(acrylamide sulphonic acid), polyacrylonitrile, poly(vinyl pyrrolidone), agar, dextran, dextrin, carrageenan, xanthan, and guar. The hydrophilic polymers can also include ionizable groups such as acid groups, e.g., carboxylic, sulphonic or nitric groups. The hydrophilic polymers may be cross-linked through a suitable cross-binding compound. The cross-binder actually-used depends on the polymer system: If the polymer system is polymerized as a free radical polymerization, a preferred cross-binder comprises 2 or 3 unsaturated double bonds. Alternatively, the lubricious coating may be any biostable hydrogel as is known in the art. Alternatively, expanded polytertrafluoroethylene (ePTFE) may be used as the hydrophilic polymeric coating. It may also contain or be coated with substances that promote endothelialization and/or retard thrombosis and/or the growth of smooth muscle cells.

The biocompatible polymers, described herein, may be applied using any technique known in the art known to one of ordinary skill in the art, including dipping. Alternatively, the polymers may be sprayed using a spray nozzle and the coating subsequently dried to remove solvent. The spraying may occur as the stent is placed onto a mandrel. The mandrel may be rotated during spraying to promote uniform coating. Any suitable rate of rotation can be used that provides uniform coating. The polymer may also be applied as a solution. If necessary, gentle heating and/or agitation, such as stirring, may be employed to cause substantial dissolution.

The coating may also include any woven material or biological material known to one of ordinary skill in the art.

A variety of factors may be considered in determining a suitable thickness for the coating 108, including the implantation site, the particular configuration of the zigzag cages, the braided pattern of the hybrid stent 100, and the tendency of the hybrid stent 100 to kink. In the particular embodiment of FIG. 1, the thickness of the coating may range from about 0.030 mm to about 0.60 mm. Determination of a suitable thickness of the coating 108, based upon the above factors, can be determined by one of ordinary skill in the art.

Still referring to FIG. 1, because the coating 108 connects the proximal, distal portions 105, 106 and the body portion 112 to each other, no interconnectors within the hybrid stent 100 are required. Accordingly, gaps L₁ and L₂ are shown. L₁ is the gap between the body portion 112 and the distal portion 106. L₂ is the gap between the body portion 112 and the proximal portion 105. Such gaps between the zigzag sections and the braided portion promote further flexibility of the hybrid stent 100.

Additionally, the L₁ and L₂ gaps may impart so-called dampening characteristics to the hybrid stent 100. The gaps L₁ and L₂ enable the hybrid stent 100 to oppose external forces that are typically encountered at an implantation site. For example, referring to FIG. 1, if the hybrid stent 100 is implanted within the esophagus, an external force, such as a peristaltic muscular contraction which forces food particles down through the esophagus may be encountered by the proximal portion 105. As the external force travels from the proximal direction to the distal direction, the hybrid stent 100 may have a tendency to migrate in the direction of the external force. However, the flexibility of gap L₁ and gap L₂ will dissipate the external force such that the tendency of the hybrid stent 100 to migrate is reduced. In particular, upon encountering the external force, the hybrid stent 100 will undergo an accordion-like movement in which the hybrid stent 100 will reduce in length and subsequently expand in length to dissipate the external force. Such an accordion-like movement enables the hybrid stent 100 to be flexible in a longitudinal direction, which reduces its tendency to migrate downstream from the implantation site. Such dampening characteristics are applicable in any body lumen that inherently moves. The predetermined distance of the gaps L₁ and L₂ are dependent upon numerous factors. A suitable L₁ and L₂ are preferably chosen such that kinking of the covering does not occur and effective flexibility and dampening are still enabled. Suitable ranges of the length of the gaps L₁ and L₂ include from about 0.5 mm to about 7 mm, preferably from about 1.5 mm to about 6 mm, and most preferably from about 2 mm to about 4 mm. The length of gaps L₁ and L₂ may be identical or different.

As shown in FIG. 1, because the continuous coating 108 covers the entire hybrid stent 100, tissue in-growth through the braided openings and zigzag shaped member interstices is prevented. This enables removal or repositioning of the hybrid stent 100 by way of a retrieval wire 104, shown in FIG. 1. A retrieval wire 104 is disposed at the proximal portion 105. The retrieval wire 104 may be configured in various manners. For example, the retrieval wire 104 may be sutured through the eyes 121. Forceps may be used to engage the retrieval wire 104 and subsequently remove or reposition the hybrid stent 100.

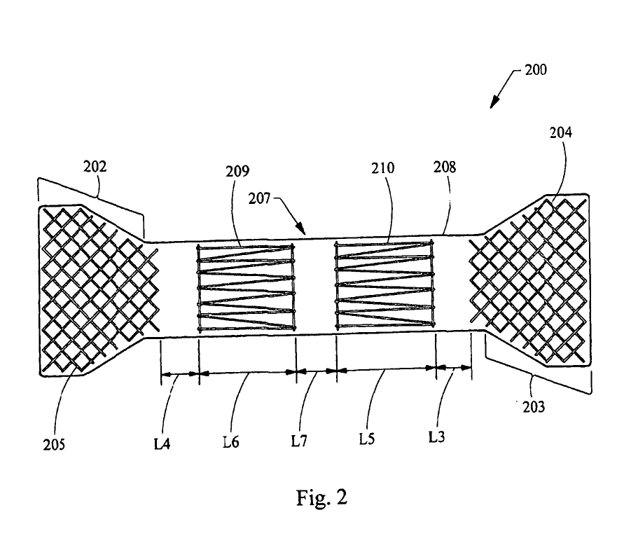

FIG. 2 shows another hybrid stent 200 with a combination of zigzag and braided stent characteristics. The hybrid stent 200 is a combination of zigzag cages 209, 210 along the body portion 207, a braided pattern stent 205 at the proximal portion 202, and a braided pattern stent 204 at the distal portion 203. Generally speaking, the combination of a braided design at the proximal portion 202 and distal portion 203 with zigzag cages along the body portion 207 results in a hybrid stent 200 that may be suitable for implantation at body lumens where a relatively low radial force is required to minimize tissue overgrowth. The problem of tissue overgrowth will be explained below.

The proximal portion 202 and distal portion 203 have respective braided patterns 205, 204. The braided patterns 205, 204 may be formed from a single wire or multiple wires. Various geometries of the proximal portion 202 and distal portion 203 are contemplated, including cup-shaped and sphere-shaped. The braided patterns 205, 204 provide a radial force against a body lumen that is relatively lower than the radial force exerted by the zigzag arrangement of hybrid stent 100, shown in FIG. 1. A lower radial force may be required for certain body lumens where surrounding tissue is susceptible to irritation. When a large radial force is exerted against such tissue to cause irritation, the tissue may respond by proliferating over a portion of the stent. Such a phenomena is called tissue overgrowth. Tissue overgrowth is problematic as it can lead to inflammation. Tissue overgrowth also prevents the hybrid stent 200 from subsequently being repositioned or removed. Accordingly, certain medical applications will benefit from a hybrid stent of the type shown in FIG. 2, in which the soft ends of the braided pattern provide a relatively smaller radial force against the tissue of the body lumen, thereby reducing the likelihood of tissue overgrowth. A smaller radial force will result in a smaller stimulus to the tissue, thereby reducing the chance of tissue overgrowth over the proximal portion 202 and/or distal portion 203 of the hybrid stent 200.

As shown and described with respect to the hybrid stent 100 of FIG. 1, hybrid stent 200 also has an overlying coating 208 that is continuous along the entire length. FIG. 2 shows that the coating 208 completely covers the hybrid stent 200 such that tissue in-growth may be prevented. The coating may be any biocompatible material such as THORALON. Predetermined gaps L₄ and L₃ provide flexibility and dampening characteristics. L₃ is the gap between zigzag cage 210 and distal portion 203. L₄ is the gap between zigzag cage 209 and proximal portion 202. A suitable length for L₃ and L₄ is preferably chosen such that kinking of the covering does not occur and effective flexibility and dampening is still enabled. Suitable ranges of the length of the gaps L₁ and L₂ include from about 0.5 mm to about 7 mm, preferably from about 1.5 mm to about 6 mm, and most preferably from about 2 mm to about 3 mm. The length of gaps L₃ and L₄ may be identical or different.

The body portion 207 includes zigzag cage 209 and zigzag cage 210 spaced apart at a predetermined distance, L₇. Although FIG. 2 shows two zigzag cages disposed within the body portion 207, more than or less than two zigzag cages may be used. A suitable number of zigzag cages is dependent upon many factors, including the length of the stricture and the implantation site. The use of zigzag cages within the body portion 207 provides a large radial force, which can dilate the body lumen that the hybrid stent 200 is placed within.

Zigzag cages 209, 210 are attached to the coating 208. The absence of any interconnectors between zigzag cage 209 and zigzag cage 210 reduces the stiffness and rigidity normally associated with zigzag structures. Accordingly, zigzag cage 209 is shown to be spaced apart from zigzag cage 210 by a gap L₇. Gap L₇ imparts flexibility and the capability of the body portion 207 to flex within curved vasculature and body lumens. A suitable length for L₇ may be chosen such that kinking of the covering does not occur and flexibility and dampening of the hybrid stent 200 is permitted. Suitable ranges of the length of the gaps L₇ include from about 0.5 mm to about 5 mm, preferably from about 1 mm to about 4 mm, and most preferably from about 2 mm to about 3 mm.

A suitable length for each of the zigzag cages 209, 210, denoted as L₅ and L₆ in FIG. 2, may primarily be dependent upon the length of the stricture that the zigzag cages 209, 210 will contact. In the example shown in FIG. 2, the lengths of the zigzag cages 209 and 210, L₅ and L₆, are each about 20 mm. Although both zigzag cages 209, 210 have an identical length, the zigzag cages may have different lengths. The zigzag cages may be made from any biocompatible material, including stainless steel and nitinol. Other metallic alloys and shape memory metals are contemplated.

Similar to the retrieval wire 104 shown in FIG. 1, hybrid stent 200 may also contain a retrieval wire at its proximal portion 202. Accordingly, a retrieval device may be used to engage the retrieval wire for removal or repositioning of the hybrid stent 200.

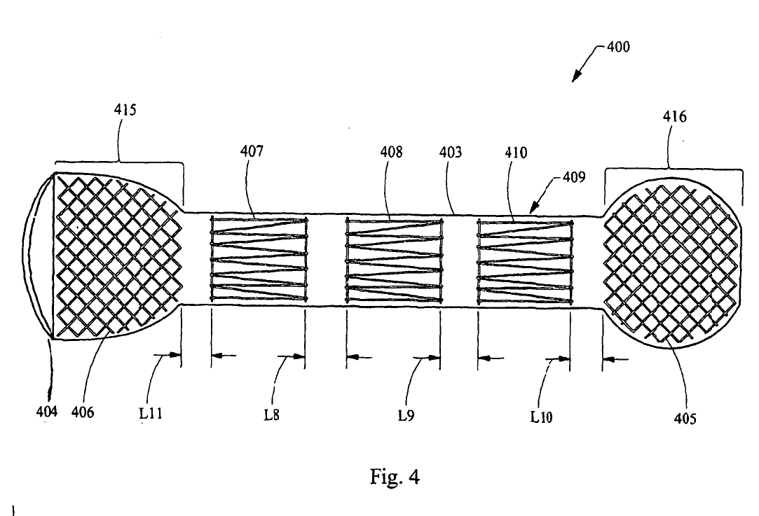

FIG. 4 shows another hybrid stent 400. The hybrid stent 400 has three zigzag cages, thereby making the body portion 409 of hybrid stent 400 longer in length than the body portion of hybrid stent 207 of FIG. 2. Accordingly, if a relatively large radial force is desired in the body portion, and the length of the stricture is relatively long, hybrid stent 400 may be a more viable selection over hybrid stent 200 of FIG. 2. Zigzag cages 407, 408, and 410 are disposed within the body portion 409. Zigzag cage 408 is spaced apart a predetermined distance L₈ from zigzag cage 407 and zigzag cage 408 is spaced apart a predetermined distance L₈ from zigzag cage 410. In this example, L₈ and L₉ each have a distance ranging from about 2 mm to about 4 mm. Other distances for L₈ and L₉ may be used and are dependent upon numerous factors, including the length of the stricture and the implantation site. In this example, the length of each zigzag cage is about 20 mm. Depending at least partially on the length of the stricture and implantation site, other lengths of zigzag cages may be used.

Hybrid stent 400 has a proximal portion 415 which has braided pattern 406 and a distal portion 416 which has braided pattern 405. Braided patterns 405 and 406 may be formed from a single wire or multiple wires. Braided patterns 405 and 406 may have identical or different braid sizes. With respect to the geometry, proximal and distal portions 415 and 416 are shown to have a flared shape. In particular, proximal portion 415 is cup-shaped. The geometry provides a radial force which is sufficient to prevent migration of the hybrid stent 400. Distal portion 416 is sphere-shaped. Such a sphere-shape renders anatomical compatibility when the implantation site is the esophagus. Anatomical compatibility with the esophagus reduces the possibility of perforation and bleeding of tissue that the sphere-shaped distal portion 416 contacts. The radial force exerted by the sphere-shaped distal portion 416 is less than the radial force exerted by the cup-shaped distal portion 416. Other embodiments are contemplated in which the radial force at the proximal and distal portions of the stent may be identical or in which the radial force at the distal portion may be greater than the radial force at the proximal portion.

In this example of FIG. 4, both the cup-shaped proximal portion 415 and the sphere-shape distal portion 416 are fabricated with nitinol wire. Additional suitable biocompatible materials are contemplated, including stainless steel, various metallic alloys, and other shape memory metals besides nitinol. Similar to hybrid stent 100 of FIG. 1 and hybrid stent 200 of FIG. 2, a continuous coating 403 is disposed over the zigzag cages 407, 408, 410 and the braided flared ends of proximal and distal portions 415, 416. The continuous coating 403 reduces tissue in-growth. The coating 403 also eliminates the need to interconnect each of the zigzag cages 407, 408, 410 with each other and the proximal portion 415 with zigzag cage 407 and the distal portion 416 with zigzag cage 410. Accordingly, greater flexibility is imparted to the body portion 409 of hybrid stent 400 than would result if a typical zigzag was used. The gaps between proximal portion 415 and zigzag cage 407, L₁₁, and between distal portion 416 and zigzag cage 410, L₁₀, impart flexibility to the hybrid stent 400. All of the gaps, L₈-L₁₁, promote flexibility during delivery and deployment at the implantation site as well as dampening of external forces encountered in moving body lumens.

Because the coating 403 is continuous and extends the entire length of the hybrid stent 400, tissue in-growth is prevented. A retrieval wire 404 may be configured about the proximal portion 415 of hybrid stent 400. A retrieval device may be introduced to engage the retrieval wire 404 and reposition the hybrid stent 400 at another implantation site. Alternatively, the retrieval device may engage the retrieval wire 404 for the purpose of withdrawing the hybrid stent 400 from the patient's body.

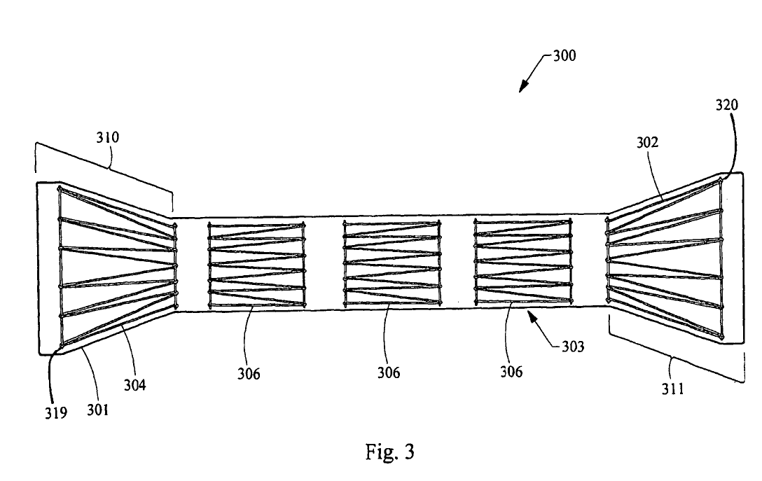

FIG. 3 shows a hybrid zigzag 300 that may be used in applications where tissue overgrowth is a concern. Tissue overgrowth, also known as hyperplasia, is the growth of healthy tissue around the ends of the stent. Hyperplasia may occur when the ends of the stent exert an excessive radial force on the normal tissue which stimulates the endothelial cells of the normal tissue to grow. Typically, zigzags exert a relatively high radial force against the body lumens they are implanted within. Sensitive body tissue may become irritated by such a high radial force and respond by tissue overgrowth around one or both ends of the zigzag. However, the hybrid zigzag 300 possesses lower radial force at the ends for reasons that will now be discussed.

The hybrid zigzag 300 has thinner diameter wire 304, 302 at the respective proximal and distal portions 310, 311 than at the body portion 303. Because a larger wire diameter yields a greater radial force, the thinner diameter wire 304, 302 may produce a radial force that is smaller at the proximal and distal portions 310, 311 than at the body portion 303. In this example, proximal portion 310 uses stainless steel wire 304 having a wire diameter of about 0.011 inches. Distal portion 311 also uses stainless steel wire 302 having a wire diameter of about 0.011 inches. The body portion 303 uses wire diameter 306 having a diameter of about 0.015 inches. Other wire diameters may be used along the proximal and distal portions 310, 311 and the body portion 303.

In addition to utilizing larger diameter wire for each of the three zgzag cages along the body portion shown in FIG. 3, other wire properties may be altered to achieve a smaller radial force at the proximal and distal portions 310, 311 relative to the body portion 303. For example, the larger radial force along the body portion 303 may be achieved by increasing the number of zigzag elements that each of the three zigzag cages possess along the body portion 303 compared to the number of zigzag elements for the zigzag cage of the proximal portion 310 and the zigzag cage of the distal portion 311. The number of zigzag elements for a zigzag cage as used herein refers to the number of zigzag elements disposed three hundred sixty degrees about the circumference of the zigzag cage.

A coating 301 is also shown in FIG. 3. The coating 301 extends a predetermined distance beyond the proximal end 319 and the distal end 320. The coating 301 softens the proximal and distal ends 319, 320 of the zigzag cages such that tissue irritation is reduced. Reduction in tissue irritation leads to a reduction in tissue overgrowth around the proximal portion 310 and distal portion 311. The combination of varying wire properties and an extended coating will enable use of a hybrid zigzag structure having a series of zigzag cages in body lumens that typically would become irritated by an expanded zigzag.

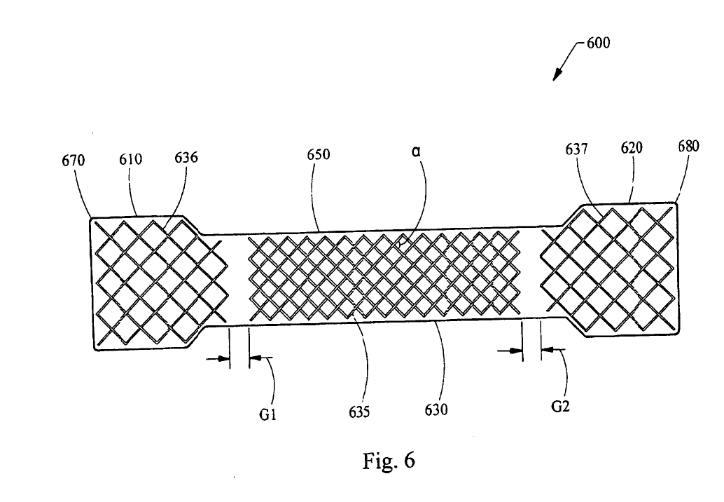

Other hybrid stent structures having variable radial force along their length may be used to minimize tissue overgrowth and tissue perforation of healthy tissue. FIG. 6 shows an example of a braided hybrid stent 600 having a first end cage 610, a second end cage 620, and a body cage 630 located between the first and second end cages 610 and 620. The body cage 630 of the braided hybrid stent 600 may be designed to exert a larger radial force than the first end cage 610 and second end cage 620. The relatively larger radial force exerted by the body cage 630 may be achieved by utilizing braid elements 635 that possess a larger diameter and larger crown number compared to braid elements elements 636 and 637 of the first end cage 610 and second end cage 620, respectively. The crown number as defined herein refers to the number of braid elements per unit area within a cage. Even though the first end cage 610 and second end cage 620 have a larger diameter than the body cage 630, the smaller wire diameter and crown number of braid elements 636 and 637 offset the increase in radial force caused by larger diameter ends. Thus, the result is a stent structure which alleviates tissue perforation at the ends while still maintaining adequate radial force because of the larger diameter ends to fixate the stent 600 at a stenosed site.

In order to further reduce tissue irritation, the first and second cages 610 and 620 have ends 670 and 680 that are inwardly rounded a predetermined amount. The inward rounding is quantified by a radius of curvature. The radius of curvature may vary from about 0.5 mm to about 4 mm, preferably from about 1 mm to about 3.5 mm, and more preferably from about 1.5 mm to about 3 mm. The inward rounding of the ends 670, 680 creates a softer end which may decrease tissue irritation, thereby reducing the occurrence of tissue overgrowth around the ends 670 and 680 of the stent. The braided hybrid stent 600 shown in FIG. 6 is completely covered by a polymeric covering 650 to prevent tissue in growth through the braidses of the stent 600. The polymeric covering 650 is shown as completely circumscribing the stent 600 and extending continuously in the longitudinal direction from inwardly rounded end 670 to inwardly rounded end 680. Covering 650 may alternatively only partially cover the cages of stent 600.

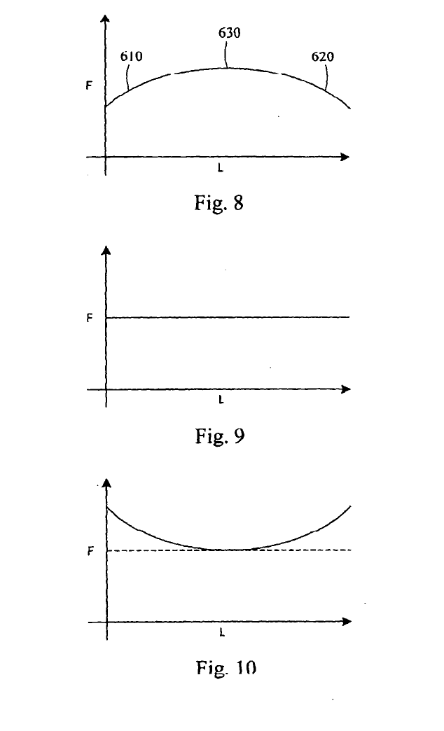

The distribution of outward radial force exerted against a body lumen along the length of the stent 600 of FIG. 6 may be plotted as shown in FIG. 8. FIG. 8 shows that the body cage 630 exerts greater force than the first end cage 610 and the second end cage 620. The body cage 630 exerts a sufficiently high radial force against the endothelial tissue cells of the stenosed region to stimulate it to rapidly grow, as desired. The first end cage 610 and the second end cage 620 exert a relatively low force, as compared to the body cage 630, that does not allow the endothelial tissue cells to be stimulated to grow quickly, as desired. This force distribution is favorable compared to typical stents which may have a force distribution as shown in FIGS. 9 or 10. FIG. 9 shows the outward radial force distribution that may be exerted by a stent having a uniform stent structure along the length thereof FIG. 9 shows that the outward radial force that is exerted against a body lumen is substantially constant over the length of the stent. The relatively larger force at the ends in FIG. 9, as compared to FIG. 8, may cause tissue overgrowth around the ends of the stent as well as tissue perforation. FIG. 10 is an alternative force distribution of a typical stent. In particular, FIG. 10 illustrates the outward radial force distribution that may be exerted by a stent having a uniform stent structure along the length thereof, and also having larger diameter ends (compared to the middle, body section). Because the elements of the ends have the same diameter and same crown number as the elements in the middle, the elements at the ends will exert a larger radial force, as shown in the plot of FIG. 10. As a result, the higher radial force exerted at the ends may cause significant tissue perforation and tissue overgrowth.

Although variation in crown number and wire diameter have been described as the means to achieve radial force variation along the length of a stent, other means are contemplated. For example, referring to FIG. 6, the hybrid braided stent 600 may possess braid angle variation. The braid angle being referred to herein is the braid angle along the longitudinal axis of the stent in its relaxed state, which is labeled as α in FIG. 6. Generally speaking, a smaller braid angle allows the stent to expand more than a stent with a larger braid angle. Thus, the smaller the braid angle, the bigger the radial force. Therefore, although not shown in FIG. 6, a smaller braid angle within the body cage 630 as compared to the first and second cages 610, 620 will create a stent that exhibits a larger radial force at the body cage 630 relative to the first and second end cages 610, 620. The precise angle will depend on a variety of factors, including the implantation site. For example, a stent that is to be implanted within the duodenum or colon needs to be more flexible than an esophageal stent. The primary reason is because of the greater inherent tortuosity present within the duodenum and colon as compared to the esophagus. Accordingly, the braid angle should be smaller than that used in a typical esophageal stent because a small braid angle provides greater flexibility.

Still referring to FIG. 6, the preferred dimensions of the braided hybrid stent 600 are as follows. The longitudinal length of the body cage 630 should be sufficient to extend along the entire length of the stenosed region. Suitable longitudinal lengths for the body cage 630 may range between about 40 mm to about 200 mm. The diameter of the body cage 630 should likewise be sufficient to contact the entire stenosed region when the stent 600 has expanded. Suitable diameters for the body cage 630 may range between about 15 mm to about 25 mm. For esophageal applications, the diameter may preferably be closer to the lower range. For duodenum and colonic applications, the diameter may preferably be closer to the upper range. The first end cage 610 and second end cage 620 are each designed to be larger than the body cage 630 by a predetermined amount such that the stent 600 will be able to remain fixated at the stenosed region. Preferably, the first and second end cages 610, 620 will have a diameter that is between 5 mm to about 8 mm larger than the diameter of the body cage 630.

Gaps G₁ and G₂ are designed to promote adequate flexibility and pushability of the stent 600. The gaps G₁ and G₂ enable the stent to flex when encountering a curved lumen. Generally speaking, a larger gap assists in increased flexibility and a smaller gap assists in improved pushability. The length of the gap G₁ and G₂ may vary between about 2 mm to about 4 mm. The size of the gap is dependent upon the stent diameter. As an example, if the diameter of the body cage 630 is relatively small (e.g., 15 mm), then the gap may preferably be as small as 2 mm in order to provide adequate flexibility and pushability.

The braided stent structure 600 has first and second end cages 610, 620 that may be characterized as flanged shape. The flanged shape first and second end cages 610, 620 have a relatively sharp transition from the diameter of the body cage 630 to the diameter of the end cages 610, 620. When the braided stent 600 is implanted within a body lumen, the body cage 630 of the stent 600 may contact and extend the length of the stricture, and the first and second end cages 610, 620 may be in contact with healthy tissue adjacent to the stricture. The diameter of the flanged shape first and second end cages 610, 620 may be sufficient to maintain fixation of the stent 600 but yet not large enough to exert a radial force that perforates the tissue and/or causes tissue overgrowth around the first and second end cages 610, 620.

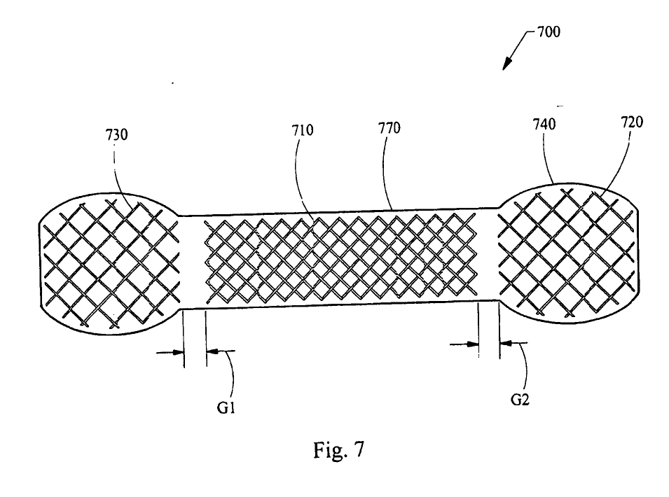

Various alternative shaped first and second end cages are contemplated. For example, the end cages may be flared such that the transition in diameter from the body cage 630 to the end cages 610, 620 is gradual and continuous. FIG. 7 illustrates another example and is a preferred embodiment. Specifically, FIG. 7 shows a braided hybrid stent 700 with dumbbell-shaped first end cage 730 and dumbbell-shaped second end cage 740. The dumbbell-shaped end cages 730, 740 may be characterized by a gradual increase in diameter from the body cage 710 to the mid-point of the end cages 730, 740 followed by a gradual taper towards the ends of the end cages 730, 740. The dumbbell shaped ends may have the ability to fixate the stents without causing tissue perforation and tissue overgrowth. Similar to the braided hybrid stent 600 of FIG. 6, the braid elements of the first end cage 730 and second end cage 740 may have a smaller crown number and a smaller diameter than the braid elements of the body cage 710 for the purpose of reducing the outwardly directed radial force, thus minimizing the likelihood of tissue perforation and tissue overgrowth.

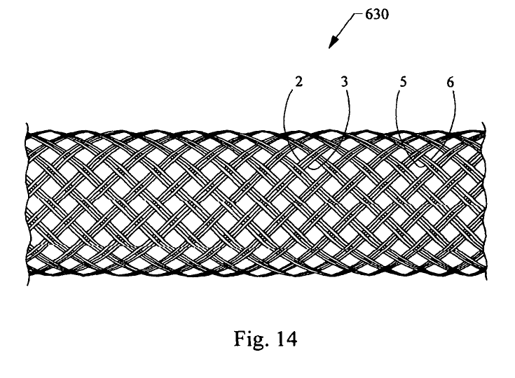

Increasing the crown number (ie., the number of wire elements per unit area), has been discussed as one of the ways to provide higher radial force at the body cage relative to the end cages. FIG. 14 exhibits one way of achieving a higher crown number at the body cage. FIG. 14 shows the body cage 630 of FIG. 6 in which a first pair of filaments 2, 3 are wound in one helical direction and another pair of filaments 5, 6 are wound in a second helical direction. Filament 2 and 3 are arranged side by side. Filament 5 and 6 are likewise arranged side by side. Although a pair of filaments are shown extending in each of the first and second helical directions, three, four or more filaments may be provided which extend in each of the first and the second helical directions. Alternatively, additional thread elements may be separately interlaced along the body cage to create the desired interlacing density of braid elements along the body cage 630.

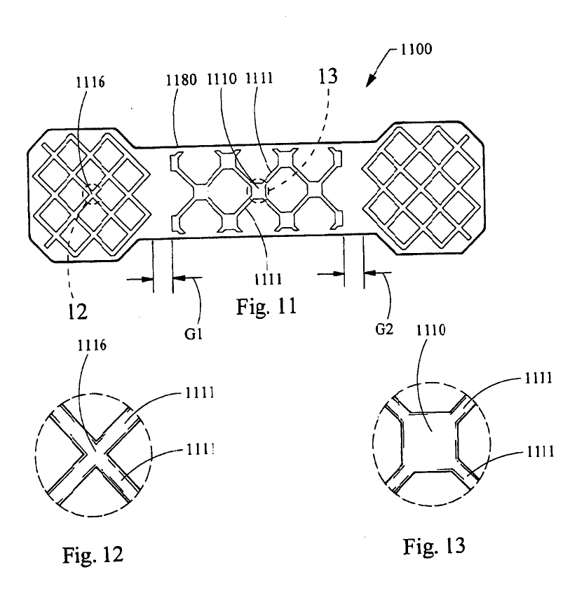

Other hybrid stent structures may be utilized to create a radial force that is greater along the middle section of the stent than at the end sections without causing the stent to migrate from the stenosed region. In one example, a tubular stent structure 1100 as shown in FIGS. 11-13 is formed of elements meeting at junctions 1116 and 1110, where the junction size can be varied along different portions of the stent 1100. Stent 1100 is shown including braid elements 1111. Braid elements 1111 intersect each other at junction 1116 as shown in FIG. 12 and at junction 1110 as shown in FIG. 13. FIG. 13 illustrates a junction 1110 having a greater amount of material than the junction 1116 of FIG. 12. The junctions are cut in the expanded deployed configuration. Thus, junctions having more material (i.e., greater surface area) have greater resistance to flex from the outward bias position, and therefore greater capacity to provide radial outward force than junctions having less material (i.e., less surface area). The junctions 1110 and 1116 may be formed by laser cutting a nitinol tube material. Thus, the braid elements 1111 at the body section of the stent may be capable of exerting a larger outward radial force than the braid elements 1111 at the end sections of the stent. The result is a larger radial force along the stenosed region and less radial force at the end sections which are in contact with healthy tissue. Although the flanged shape sections exert less radial force than the center section, the flanged shape end sections nevertheless have a sufficient diameter and geometry to prevent migration of the stent 1100 from the stenosed region. Additionally, the gaps G₁ and G₂ assist in the dampening of external forces encountered by the stent in implantation sites such as the esophagus where peristaltic forces occur. Thus, the gaps G₁ and G₂ between the cage structures may assist in the prevention of migration of the stent 1100.

Various shapes of the wires may be used. Differing wire shapes enable the radial force that is against a body lumen to be varied as desired. For example, a flat wire may in certain applications be preferable over a circular-shaped cross-sectional wire. The flat shaped wire may be suitable for use along the body cage of the hybrid stent where an increase in radial force is desired.

Any combination of the above-described design variables may be utilized to produce a stent structure in which the body section exerts a larger radial force than the end sections with the end sections still being capable of fixating the stent within a target site. Other hybrid stent structures may be utilized to create a radial force that is greater along the middle section of the stent than at the end sections without causing the stent to migrate from the stenosed region. These structures include serpentine configured stents, coiled stents, and zigzag shaped stents, the zigzag shaped configuration having been discussed above in conjunction with FIG. 3.

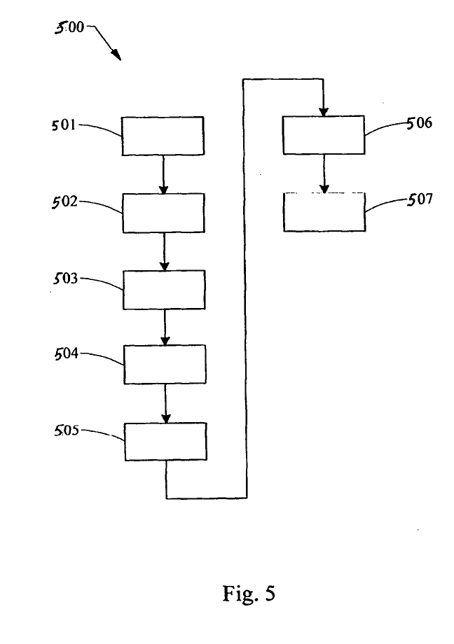

FIG. 5 shows a flow schematic outlining the steps of a fabrication process 500 of a hybrid stent. In the first step 501, the body portion and proximal and distal portions are mounted onto a smooth mandrel. The body portion may include one or more zigzag cages and the proximal and distal portions may be braided stents. Alternatively, the body portion may be a woven stent and the proximal and distal portions may be zigzag shaped stents. Alternatively, the body portion may include a series of zigzag cages and the proximal and distal portions may include another set of zigzag cages, wherein the zigzag cages of the body portion comprise a larger wire diameter, crown number, smaller braid angle, or any combination thereof compared to the end portions. The body portion and end portions may include a series of braided stents.

In step 502, all of the components are spaced apart at their predetermined distances. With all of the components on the mandrel, the proximal and distal portions are selectively placed a predetermined distance apart from the body portion. This distance will be the gaps L₁ and L₂ (FIG. 1) that the final hybrid stent will attain. If the body portion contains a series of zigzag cages, then each of the zigzag cages are separated at their respective predetermined distances. This distance between the zigzag cages will be the gaps L₈ and L₉ (FIG. 4) that the final hybrid stent will yield. Any number of zigzag cages are contemplated within the body portion.

The components are in their expanded state. Step 503 involves maintaining the components at their selected position on the mandrel. A number of different ways for maintaining the positioning of the components is contemplated. For example, if zigzag cages are utilized, each of the zigzag cages may have a retrieval wire on their respective proximal and distal ends that may be pulled. Alternatively, the zigzag cages may be soldered together for the purpose of maintaining the desired spacing of the zigzag cages on the mandrel. Other suitable means of maintaining the shape of the zigzag cages and the braided cages on the mandrel, including suturing and tying together the cages may be utilized as known to one of ordinary skill in the art.

After all the components have been placed in their selected positions on the mandrel, step 504 involves coating the whole mandrel assembly with a polymer. Suitable ways of coating the polymer onto the mandrel assembly are known to one of ordinary skill in the art. For example, the polymer may be sprayed onto the mandrel assembly. Preferably, the polymer is dip coated into a polymer solution.

In step 505, the mandrel assembly is removed from the polymer solution after sufficient time has elapsed for the coating to fill all the interstices of the zigzags and/or braids.

In step 506, the mandrel assembly is allowed suitable time for the polymer to dry. The polymer will not stick on the surface of the mandrel. Rather, it will adhere to the surfaces of the zigzag cages and/or braided stent, thereby connecting all of the components. Upon drying, the individual components form an integrated stent assembly known as the hybrid stent.

After the polymer has dried, step 507 comprises removing the mandrel from the hybrid stent. Because the mandrel is smooth and possesses a low coefficient of friction, the mandrel may readily be removed from the hybrid stent.

The above figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

1. An intraluminal device, comprising:
a first expandable stent structure;
a second expandable stent structure, wherein said second expandable stent structure is separated by at least one predetermined gap, said at least one predetermined gap being sufficient to dampen external forces encountered by said device in an implanted lumen; and
a coating attached to said first expandable stent structure and said second expandable stent structure, wherein said first expandable stent structure comprises a radial expanding force that is different than said second expandable stent structure.

2. The intraluminal device of claim 1, wherein said first expandable stent structure comprises a first stent structure, said first stent structure comprising one or more first elements, further wherein said second expandable stent structure comprises a second stent structure, said second stent structure comprising one or more second elements, wherein said first stent structure is different from said second stent structure.

3. The intraluminal device of claim 2, wherein said one or more first elements comprises a first diameter and said one or more second elements comprises a second diameter, said first diameter being different from said second diameter.

4. The intraluminal device of claim 3, wherein the one or more first elements form a body cage and the one or more second elements form an end cage.

5. The intraluminal device of claim 4, wherein the radial expanding force of the body cage is greater than the radial expanding force of the end cage.

6. The intraluminal device of claim 5, wherein the first diameter is greater than the second diameter.

7. The intraluminal device of claim 5, wherein the body cage comprises a number of the one or more first elements per unit area greater than the number of the one or more second elements of the end cage per unit area.

8. The intraluminal device of claim 5, wherein the body cage comprises a braid angle that is smaller than a braid angle of the end cage.

9. The intraluminal device of claim 5, wherein the one or more first elements has a first diameter greater than a second diameter of the one or more second elements, a greater number of the one or more first elements per unit area than the number of the one or more second elements per unit area, a braid angle of the one or more first elements smaller than a braid angle of the one or more second elements, or any combination thereof.

10. The intraluminal device of claim 5, wherein the end cage is one of flared shape, flanged shape, and dumbbell shaped, further wherein the end cage has a larger diameter than the body cage, the larger diameter of the end cage being sufficient to substantially prevent migration of the device from an implanted site.

11. The intraluminal device of claim 7, wherein the one or more first elements of the body cage comprises two or more first filaments wound together in a first helical direction along a longitudinal axis of the device and two or more second filaments wound together in a second helical direction along the longitudinal axis.

12. The intraluminal device of claim 7, wherein the one or more first elements of the body cage comprises one or more wires separately interlaced along the body cage to create an interlacing density along the body cage.

13. The intraluminal device of claim 5, wherein the one or more second elements of the end cage intersect to form a first plurality of junctions, further wherein the one or more first elements of the body cage intersect to form a second plurality of junctions, the first plurality of junctions having less surface area than the second plurality of junctions.

14. The intraluminal device of claim 5, wherein the body cage comprises variation in radial expansive force along a longitudinal length of the body cage.

15. The intraluminal device of claim 5, wherein the one or more first elements of the body cage and the one or more second elements of the end cage form one of a braided structure, zigzag structure, serpentine structure, and coiled structure.

16. The intraluminal device of claim 19, wherein said one or more first elements of a first arrangement further comprises a zigzag shaped configured stent and said one or more second elements of a second arrangement further comprises a braided configured stent.

17. The intraluminal device of claim 1, wherein said coating extends a predetermined distance beyond one of said first expandable stent structure and said second expandable stent structure of said device, said distance being sufficient to reduce tissue overgrowth.

18. The intraluminal device of claim 1, wherein said coating is formed from a drug eluting polymer carrier, said drug eluting polymer carrier being loaded with one or more bioactives.

19. The intraluminal device of claim 2, wherein said first stent structure comprises a body portion of the device, said body portion including one or more first zigzag shaped stents having a first wire diameter, further wherein said second stent structure comprises a first end portion and a second end portion, said first and second end portions comprising one or more second zigzag shaped stents having a second wire diameter, said second wire diameter being different from said first wire diameter.

20. The intraluminal device of claim 19, wherein said body portion comprises a first wire material and a first wire form density, further wherein said first and second end portions comprise a second wire material and a second wire form density, said first wire material different from said second wire material, and said first wire form density being different from said second wire form density.

21. An intraluminal device, comprising:
a body portion, said body portion further comprising a plurality of zigzag shaped stents having an outer body diameter, wherein each of said plurality of zigzag shaped stents are longitudinally spaced apart without being interconnected to each other, wherein each of said plurality of zigzag shaped stents extend circumferentially around a generally cylindrical body and extend along at least a portion of a longitudinal axis of said cylindrical body;
a first end portion and a second end portion, one of said first and second end portions further comprising a flexible element, one of said first and second end portions having an outermost diameter greater than said outer body diameter of said plurality of zigzag shaped stents, said first and second end portions extending in a helical pattern along at least a portion of said longitudinal axis to form a braided configuration, said braided configuration being interwoven; and
a coating attached to said body portion and said first and second end portions.

22. The intraluminal device of claim 21, wherein said plurality of zigzag shaped stents are longitudinally spaced apart a predetermined distance, said predetermined distance being sufficient to provide flexibility and maneuverability of the device in curved vasculature.

23. The intraluminal device of claim 21, wherein said body portion and one of said first and second end portions are separated by a predetermined gap sufficient to dampen external forces encountered by the device in an implanted state within a body lumen.

24. An intraluminal device, comprising:
a body portion comprising a flexible element extending in a helical pattern along at least a portion of a longitudinal axis of a cylindrical body to form a braided configuration, said braided configuration being interwoven,
a first end portion and a second end portion comprising zigzag shaped structural members disposed circumferentially around said cylindrical body and extending along at least a portion of said longitudinal axis of said cylindrical body, one of said first and second end portions having an outer diameter greater than said body portion; and
a coating attached to said body portion and said first and second end portions.

25. The intraluminal device of claim 24, wherein said zigzag shaped structural members of one of said first and second end portions engage a body lumen with a predetermined radial force sufficient to prevent migration of the device.

26. The intraluminal device of claim 24, wherein said coating extends a predetermined distance beyond one of said first and second end portions, said predetermined distance being sufficient to reduce tissue overgrowth.

27. The intraluminal device of claim 24, wherein said body portion and one of said first and second end portions are separated by at least one predetermined gap sufficient to dampen external forces encountered by said device in an implanted state within a body lumen.
